(19) United States
(12) Patent Application Publication
(10) Pub. No.: US 2007/0066870 A1
(43) Pub. Date: Mar. 22, 2007

(54) Hood for endoscope

(76) Inventors: Katsuaki Ohashi, Saitama-shi (JP); Shota Hirano, Saitama-shi (JP)

Correspondence Address: YOUNG & THOMPSON, 745 SOUTH 23RD STREET, 2ND FLOOR, ARLINGTON, VA, 22202, US

(21) Appl. No.: 11/523,617

(22) Filed: Sep. 20, 2006

(30) Foreign Application Priority Data
Sep. 22, 2005 (JP) 2005-275517
Sep. 22, 2005 (JP) 2005-275518

(51) Int. Cl. A61B 1/04

(52) U.S. Cl. 600/127; 600/129

(57) ABSTRACT

An endoscope hood for holding a medical treatment instrument inserted through an insertion channel of an insertion section of an endoscope comprises a base body portion which is capable of being fitted onto a distal end portion of the insertion section and a retainer body portion which is formed as an integral piece with the base body portion and shaped to unshroud an observation window and a lighting window and in which a predetermined length of through bore is formed so as to be in alignment with an insertion channel opening when the base portion is fitted onto the disal end portion. The retainer body portion retains a distal end portion of the medical treatment instrument inserted in the through bore through the insertion channel opening.

### BACKGROUND OF THE INVENTION

1. Field of the Invention

The present invention relates to a hood for an endoscope and, more particular, to an endoscope hood that is attached to an endoscope for holding a medical treatment instrument in order to assist a physician who observes an effected area of a patient in operating the medical treatment instrument concurrently.

2. Description of Related Art

Generally, as shown in FIG. 14, an endoscope has a distal end portion 1 provided with a lighting window 2 and an observation window 3. The endoscope comprises a light guide bundle 4 extending to the lighting window 2 and an optical lens system 5 disposed just behind the observation window 4 and has an instrument insertion channel 7 through which a medical treatment instrument such as a diathermy or high-frequency knife 6 is inserted and guided into a body cavity. This type of endoscope sends out light through the light guide bundle 4 to illuminates an observation object and focus an optical image of the observation object on a solid-state image sensing device such as CCD by the optical lens system 5. The solid-state image sensing device converts the optical image formed thereon into video signals so as to display the image on a monitor screen. A physician operates the high-frequency knife 6 inserted through the instrument insertion channel 7 and extending into a body cavity to carry out medical operation or medical treatment such as an incision and/or a resection in an affected area while watching an image of the affected area on the monitor screen.

An incident of frequent occurrence during an endoscopic treatment is unwanted moving of a treatment instrument extending out of the endoscope which works against a smooth and safety treatment of the affected area. For example, when performing an incision or a resection on an affected part by the high-frequency knife 6, the distal end portion 1 of the endoscope is moved, for example downward in FIG. 13, with an intention to bring the high-frequency knife 6 toward or in contact with the affected part. At this time, the high-frequency knife 6 is moved upward as shown by a chained line in FIG. 13 or dragged in a lengthwise direction as indicated by an arrow in FIG. 13. Consequently, the incision or the resection possibly ends up in failure.

In order to remain a treatment instrument such as a high-frequency knife stable during an endoscopic treatment, it has been proposed to use an endoscope hood for guiding a disal end portion of the treatment instrument. An endoscope hood described in, for example, Unexamined Japanese Patent Publication No. 2003-204919 comprises a hood body provided with a guide path having an approximate circular-arcuate cross section for guiding slide movement of the high-frequency knife obliquely with respect to a central axis of an instrument insertion channel. However, the endoscope hood is not good enough to restrain undesirable movement of the high-frequency knife during incision or resection in an affected area.

In addition, although a distal end portion of the high-frequency knife that protrudes from the distal end of the insertion section is monitored on the monitor screen, if the protruded portion is too short or too long, it is hard to perform accurate position control of the high-frequency knife and to allow physician to operate easily the high-frequency knife within a given field of view.

### SUMMARY OF THE INVENTION

It is therefore an object of the present invention to provide an endoscope hood capable of keeping a treatment instrument extending out from a distal end of an insertion section of an endoscope at standstill for smooth and safety endoscopic treatment.

It is another object of the present invention to provide an endoscope hood capable of keeping a predetermined length of protrusion of a treatment instrument and preventing a treatment instrument extending out from a distal end of an insertion section of an endoscope in excess safety endoscopic treatment.

The foregoing objects of the present invention are accomplished by an endoscope hood for an endoscope having an insertion section which is provided with an instrument insertion channel opening, an observation window through which an effected area of a body cavity of a patient is observed and a lighting window through which light is sent out for illuminating the effected area formed in a foreface of a disal end thereof. The endoscope hood is formed in the form of an elastic integral piece and comprises a base body portion capable of being firmly fitted onto the distal end portion of the insertion section and a retainer body portion which is formed so as to unshroud at least the observation window and the lighting window and has a predetermined length of through bore which is in alignment with the instrument insertion channel opening when the base portion is fitted onto the distal end portion of the insertion section. The retainer body portion retains a distal end portion of the medical treatment instrument inserted in the through bore through the instrument insertion channel opening.

It is desirable that the through bore has an inner diameter slightly less than an outer diameter of the distal end portion of the medical treatment instrument. It is also desirable that the retainer body is provided with stopper means, such as comprising an annular end rim formed at a distal end of the through bore so as to be engageable with a distal end of the medical treatment instrument, for preventing the medical treatment instrument from protruding in excess from the retainer body.

In the case where the endoscope hood is used in combination with a bipolar type of high-frequency knife, the endoscope hood may further comprise an electrode embedded in said retainer portion and extending from an exterior of said retainer portion to said through bore.

### BRIEF DESCRIPTION OF THE DRAWINGS

The foregoing and other objects and feature of the present invention will be clearly understood from the following detailed description when reading with reference to the accompanying drawings wherein same or similar parts or mechanisms are denoted by the same reference numerals throughout the drawings and in which:

FIG. 1A is a front view of an endoscope hood according to an embodiment of the present invention;

FIG. 1B is a side view of the endoscope hood;

FIG. 1C is a top view of the endoscope hood;

FIG. 2 is a perspective view of the endoscope hood;

FIG. 3A is a front view of a distal end of an insertion section of an endoscope;

FIG. 3B is a side view of a distal end portion of the insertion section;

FIG. 4 is a front view of the distal end of the insertion section with the endoscope hood attached thereto;

FIG. 5 is a perspective view of the distal end of the insertion section with the endoscope hood attached thereto and a treatment instrument inserted therein;

FIG. 6 is a sectional view of the distal end of the insertion section with the endoscope hood attached thereto and a treatment instrument inserted therein;

FIG. 7A is a front view of an endoscope hood according to another embodiment of the present invention;

FIG. 7B is a sectional view of the endoscope hood taken along line VII-B-VII-B of FIG. 7A;

FIG. 8A is a front view of an endoscope hood as a variant of the endoscope hood shown in FIG. 7A;

FIG. 8B is a sectional view of the endoscope hood taken along line IIX-B-IIX-B of FIG. 8A;

FIG. 9 is a perspective view of the endoscope hood shown in FIGS. 7A and 7B;

FIG. 10A is a front view of a distal end of an insertion section of an endoscope used in combination with the endoscope hood shown in FIGS. 7A and 7B or in FIG. 8A and 8B;z

FIG. 10B is a side view of a distal end portion of the insertion section;

FIG. 11 is a front view of the distal end of the insertion section with the endoscope hood attached thereto;

FIG. 12 is a perspective view of the distal end of the insertion section with the endoscope hood attached thereto;

FIG. 13 is a sectional view of the distal end of the insertion section with the endoscope hood shown in FIGS. 7A and 7B or in FIGS. 8A and 8B attached thereto and a treatment instrument inserted therein; and

FIG. 14 is a sectional view of a distal end portion of an insertion section with a prior art endoscope hood.

### DETAILED DESCRIPTION OF THE PREFERRED EMBODIMENTS

In the following description, parts or mechanisms of an endoscope which are not direct importance to the invention and parts or mechanisms of an endoscope which are purely of conventional construction will not be described in detail since their construction and operation can be easily be arrived at by those skilled in the art.

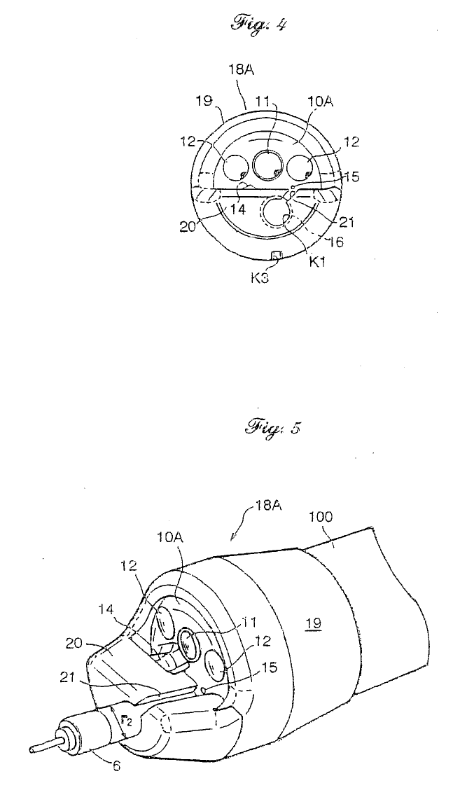

Referring to the accompanying drawings in detail, and in particular, to FIGS. 3A and 3B showing a distal end of an insertion section of an endoscope, an endoscope includes an insertions section 100 having a distal end 10A in which an observation window 11 and two lighting windows 12 on opposite sides of the observation window 11 are provided in an upper half area of the foreface of the distal end 10A. Further, there are provided an object lens system (not shown) behind the observation window 11 and light guide means (not shown) behind the respective lighting windows 12. The distal end 10A has an air/water feed nozzle 14, an injection nozzle 15 and a forceps channel opening 16 which are provided in a lower half area of the foreface of the distal end 10A. The air/water feed nozzle 14, that is connected to an air/water supply pipe extending from an operating section of the endoscope through the insertion section, splays air or water toward the observation window 11. The injection nozzle 15, that is connected to a water supply pipe extending from an operating section of the endoscope through the insertion section, sprays a jet of physiological saline or a chemical solution into a body cavity such as an affected area. A treatment instrument such a forceps, a high-frequency knife, etc. is inserted passing through a forceps channel 17 and protruded into a body cavity through the forceps channel opening 16.

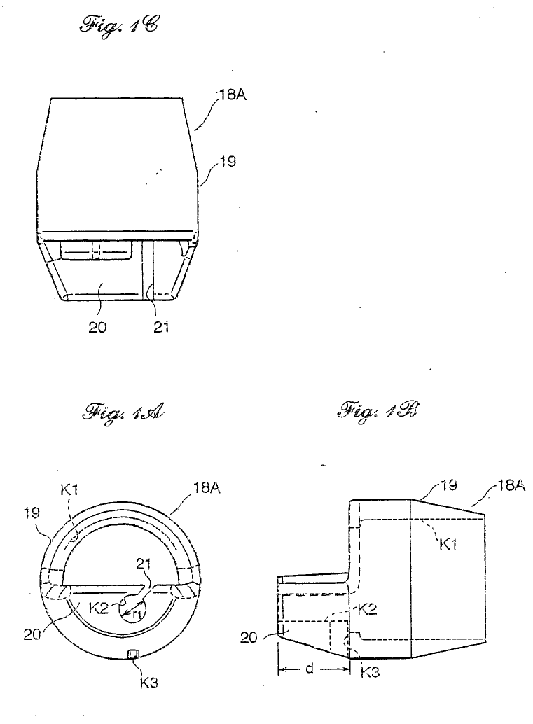

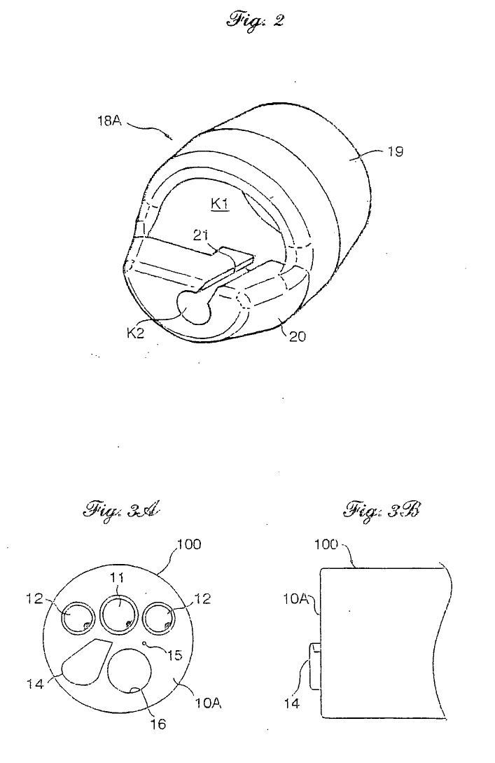

Referring to FIGS. 1A to 1C and 2 showing an endoscope hood 18A according to an embodiment of the present invention, the endoscope hood 18A is formed as an integral whippy transparent piece made of, for example, a synthetic resin such as silicone rubber. The endoscope hood 18A comprises a cylindrical main body 19 having an inner wall forming a cylindrical fitting bore K₁ and a retainer body 20 continuing from a cylindrical main body 19 which has an inner wall surface defining a cylindrical through bore K₂ having a predetermined length and is provided with a suction bore K₃ formed sideways in the wall so as to continue from the through bore K₂. The retainer body 20 has an approximately semicircular outer shape so as to take up less than half of the main body 19. Specifically, the retainer body 20 is shaped such that, when the endoscope hood 18A is attached to the distal end 10A together, it unshroud the observation window 11, the lighting windows 12 and the forceps channel opening 16 and, however, shrouds over the remaining part including the nozzle 14 of the foreface of the distal end 10A. Further, the retainer body 20 is designed to unshroud the nozzle 15 even disposed in a lower half area of the foreface of the distal end 10A as will be described later. When the endoscope hood 18A is attached to the endoscope by fitting the distal end 10A into the fitting bore K₁ of the distal end 10A together by insertion, it retains a treatment instrument, e.g. a high-frequency knife 6, inserted through the forceps channel 17 until a distal end including a needle electrode (knife) extends out of the retainer body 20 of the endoscope hood 18A, by firmly holding it in the through bore K₂. It is desirable that the retainer body 20 has a wall thickness which is substantially equal to the length d of the through bore 22, preferably in a range of from 3 to 100 mm, and more preferably of approximately 5 mm, in terms of a broad field of view and a firm grip on the high-frequency knife 6. It is also desirable that the through bore K₂ has an inner diameter r₁ approximately equal to or a little less than an outer diameter r₂ of the high-frequency knife 6. In the case where a high-frequency knife more commonly available has an outer diameter of approximately 2.4 mm, it is preferred that the through bore K₂ is 2.4 mm or less (for example 2.3 mm) in outer diameter.

As clearly shown in FIGS. 4 and 5, the retainer body 20 is provided with a slit 21 formed obliquely upward from and along the through bore K₂ in the top thereof so as thereby to unshroud the injection nozzle 15 and to allow it to spray a sharp jet of physiological saline or a chemical solution into a body cavity. Further, as was previously described and shown in FIGS. 1A, 1B and 6, the retainer body 20 is provided with the suction bore K₃ extending from the through bore K₂ so as to suck in a fluid staying in the body cavity therethrough while the high-frequency knife 6 remains in the through bore K₂.

When using the endoscope hood 18A in combination with the high-frequency knife 6 having an outer diameter r₂ approximately equal to or a little greater than an inner diameter r₁ of the through bore K₂, the endoscope hood 18A is attached to the insertions section 100 by slipping the cylindrical main body 19 on the distal end 10A as shown in FIG. 5. After having inserted the insertion section 100 with the endoscope hood 18A attached into a body cavity, the high-frequency knife 6 is inserted through the forceps channel until its front portion passes through the forceps channel opening 16 and the through bore K₂ of the endoscope hood 18A. Consequently, the high-frequency knife 6 is firmly held with the front portion extending forward from the endoscope hood 18A by a desired length as shown in FIG. 5. That is, when the high-frequency knife 6 thrust its way through the through bore K₂ of the endoscope hood 18A, the retainer body 20 is elastically deformed so as thereby to expand the through bore K₂. In this way, the high-frequency knife 6 is firmly held by the endoscope hood 18A. In this instance, the slit 21 may be formed as means for causing the through bore K₂ to be easily expanded by insertion of the high-frequency knife 6.

As shown in FIG. 6, the high-frequency knife 6 firmly held by the endoscope hood 18A is prevented from being moved up and down or from side to side, or dragged back and forth in a lengthwise direction. Further, since, in this embodiment, the retainer body 20 of the endoscope hood 18A has a wall thickness of 3 mm, which is comparatively thick, in a lengthwise direction a was previously described, the retainer body 20 keeps an assured tight hold of the high-frequency knife 6. Therefore, even when the endoscope hood 18A is made as an elastic or whippy body, the high-frequency knife 6 is prevented from being moved or dragged due to elastic deformation of the endoscope hood 18A. In addition, since the endoscope hood 18A is formed as a transparent piece, it does not stand in the way of observation of an effected area despite of a thick wall of the retainer body 20. Accordingly, a physician is enabled to perform an incision or a resection on an affected part without difficulties by using the high-frequency knife 6 in combination with endoscope hood 18A.

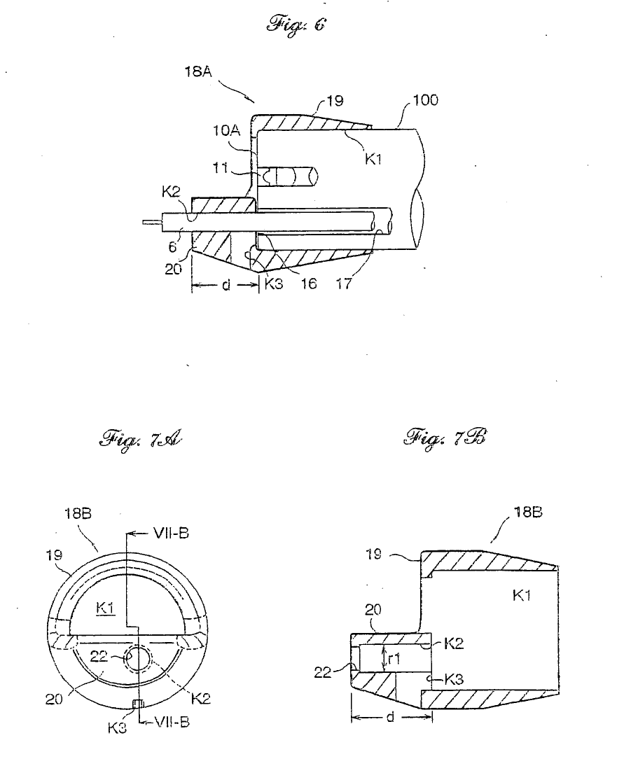

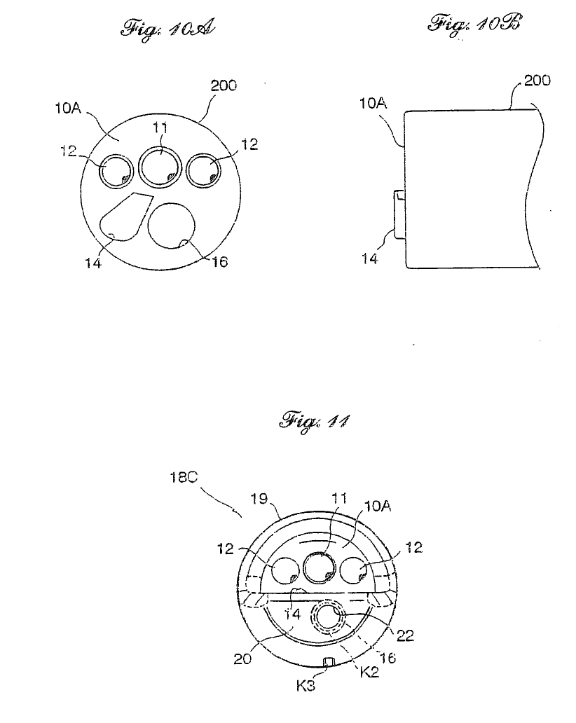

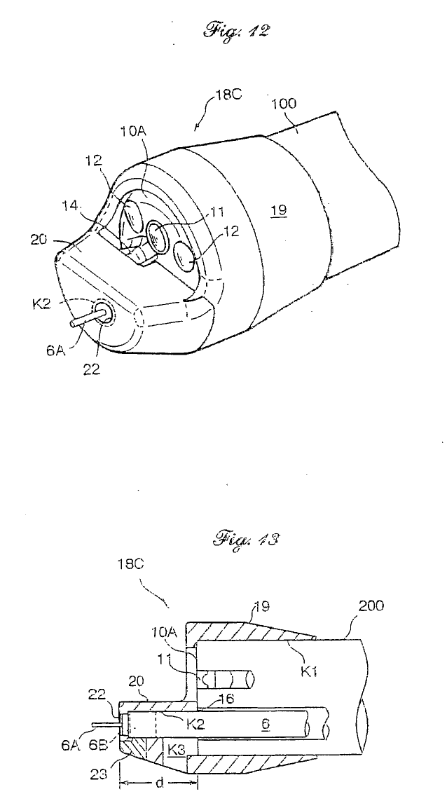

FIGS. 7A, 7B and 9 show an endoscope hood 18B, a variant of the endoscope hood 18A, according to another embodiment of the present invention for use with a high-frequency knife 6 of a monopolar type having a needle electrode (knife) 6A as shown in FIG. 13. As is well known in the art, the monopolar type high-frequency knife 6 is used in combination with an external body electrode plate (not shown) in contact with a patient body. As shown in FIGS. 10A and 10B, the endoscope includes an insertions section 200 having a disal end 10A in which an observation window 11 and two lighting windows 12 on opposite sides of the observation window 11 are provided in an upper half area of the foreface of the distal end 10A. Further, there are provided an object lens system (not shown) behind the observation window 11 and light guide means (not shown) behind the respective lighting windows 12. The distal end 10A has an air/water feed nozzle 14 and a forceps channel opening 16 which are provided in a lower half area of the foreface of the distal end 10A. The air/water feed nozzle 14 connected to an air/water supply pipe extending from an operating section of the endoscope through the insertion section splays air or water toward the observation window 11. A treatment instrument such a forceps, a high-frequency knife 6, etc. is inserted passing through a forceps channel and protruded into a body cavity through the forceps channel opening 16.

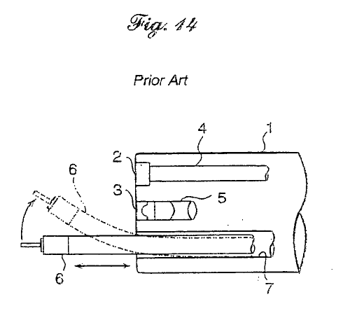

As shown in FIGS. 7A and 7B, the endoscope hood 18 is formed as an integral whippy transparent piece made of, for example, a synthetic resin such as silicone rubber. The endoscope hood 18B is almost identical in structure with the endoscope hood 18A of the previous embodiment shown in FIGS. 1A to 1C except for having an annular end rim 22 at a foremost end of a cylindrical through bore K₂ and not having a slit 21 formed obliquely upward from and along the through bore K₂ in the top of the retainer body 20 (see FIG. 1A). Specifically, the endoscope hood 18B comprises a cylindrical main body 19 having an inner wall forming a cylindrical fitting bore K₁ and a retainer body 20 continuing from a cylindrical main body 19 having a wall thickness which is substantially equal to the length d of the through bore 22, preferably in a range of from 3 to 10 mm, and more preferably of approximately 5 mm, in which a cylindrical through bore K₂ having an inner diameter of r1, approximately equal to or a little less than an outer diameter r₂ of the high-frequency knife 6, and which is provided wit an annular end rim 22 at the foremost end of the through bore K₂ and a suction bore K₃ formed sideways so as to continue from the through bore K₂. In this instance, when using the endoscope hood 18B in combination with a commonly available high-frequency knife having an outer diameter of approximately 2.4 mm, it is preferred that the through bore K₂ is 2.4 mm or less (for example 2.3 mm) in outer diameter. The retainer body 20 has an approximately semicircular outer shape so as to take up less than half of the main body 19. Specifically, the retainer body 20 is shaped such that, when the endoscope hood 18B is attached to the distal end 10A together, it unshrouds the observation window 11, the lighting windows 12 and the forceps channel opening 16 and, however, shrouds over the remaining part including the nozzle 14 of the foreface of the distal end 10A. When the endoscope hood 18B is attached to the endoscope 200 by fitting the distal end 10A into the fitting bore K₁ of the distal end 10A together by insertion as shown in FIGS. 11 and 12, it retains the high-frequency knife 6, inserted through the forceps channel opening 16 by firmly holding it in the through bore K₂. In this instance, the endoscope hood 18B prevents a needle electrode 6A (see FIG. 13) from protruding in excess from the retainer body 20 by stopping a distal end of the high-frequency knife 6 by the annular end rim 22 thereof.

The high-frequency knife 6 inserted into the endoscope is firmly held by the endoscope hood 18B and prevented from being moved up and down or from side to side, or dragged back and forth in a lengthwise direction. Further, the needle electrode 6A of the high-frequency knife 6 is prevented from protruding in excess from the endoscope hood 18B by means of abutment of the distal end of the high-frequency knife 6 against the annular end rim 22 of the endoscope hood 18B. Therefore, an affected area is prevented from brought into accidental contact with the needle electrode 6A.

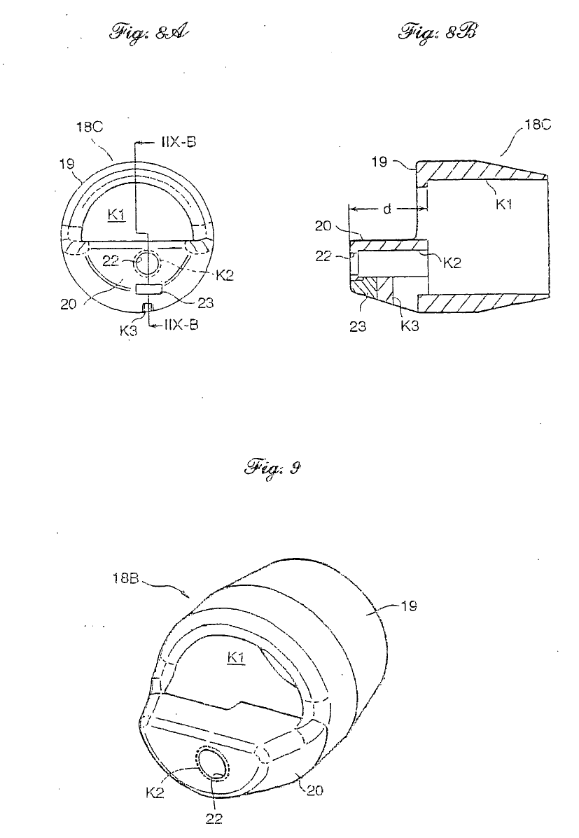

FIGS. 8A and 8B show an endoscope hood 18C, a variant of the endoscope hood 18B, according to still another embodiment of the present invention for use with a high-frequency knife 6 of a bipolar type having a needle electrode (knife) 6A and a circumferential electrode 6B (see FIG. 13). The endoscope hood 18C is identical in structure with the endoscope hood 18B of the previous embodiment shown in FIGS. 7A and 7B except for having an electrode 23. The electrode 23 is generally L-shaped and embedded in an retainer body 20 so as to extend from an exterior of the retainer body 20 into the through bore K₁ for establishing contact with the circumferential electrode 6B of the bipolar type high-frequency knife 6 when the high-frequency knife 6 is fully inserted until a distal end of the bipolar high-frequency knife 6 is stopped by an annular end rim 22 of the endoscope hood 18C as shown in FIG. 13. The high-frequency knife 6 inserted into the endoscope is firmly held by the endoscope hood 18A and prevented from being moved up and down or from side to side, or dragged back and forth in a lengthwise direction even when moving the distal end 10A of the endoscope 200 an incision or a resection on an affected part by the high-frequency knife 6. In this instance, a slit, like the slit 21 formed obliquely upward from and along the through bore K₂ in the top of the retainer body 20 of the endoscope hood shown in FIG. 1A and 1B, may be formed as means for causing the through bore K₂ to be easily expanded by insertion of the high-frequency knife 6.

It is to be understood that although the present invention has been described with regard to preferred embodiments thereof, various other embodiments and variants may occur to those skilled in the art, which are within the scope and spirit of the invention, and such other embodiments and variants are intended to be covered by the following claims.

What is claimed is:

1. An endoscope hood for an endoscope having an insertion section which is provided with an instrument insertion channel opening, an observation window through which an effected area of a body cavity of a patient is observed and a lighting window through which light is sent out for illuminating the effected area formed in a foreface of a distal end thereof said endoscope hood being attached to a distal end portion of the insertion section when the endoscope is used in combination with a medical treatment instrument, said endoscope hood comprising:
a base body portion capable of being fitted onto the distal end portion of the insertion section; and
a retainer body portion formed (as an integral piece with said base body portion) so as to unshroud at least the observation window and the lighting window, said retainer body portion having a predetermined length of through bore which is in alignment with the instrument insertion channel opening when said base portion is fitted onto the distal end portion of the insertion section;
wherein said retainer body portion retains a disal end portion of the medical treatment instrument inserted in said through bore through the instrument insertion channel opening.

2. The endoscope hood as defined in claim 1, wherein said endoscope hood is formed in the form of an elastic integral piece.

3. The endoscope hood as defined in claim 2, wherein said through bore has an inner diameter slightly less than an outer diameter of the distal end portion of the medical treatment instrument.

4. The endoscope hood as defined in claim 2, wherein said retainer body portion is provided with stopper means for preventing the medical treatment instrument from protruding in excess from said retainer body portion.

5. The endoscope hood as defined in claim 4, wherein said stopper means comprises an annular end rim formed at a distal end of said through bore so as to be engageable with a distal end of the medical treatment instrument.

6. The endoscope hood as defined in claim 4, and further comprising an electrode embedded in said retainer body portion and extending from an exterior of said retainer body portion to said through bore.
